(19) United States
(12) Patent Application Publication
(10) Pub. No.: US 2025/0360497 A1
(43) Pub. Date: Nov. 27, 2025

(54) METHOD FOR FORMING A HETERGENEOUS BIOCATALYST

(71) Applicant: REGENTS OF THE UNIVERSITY OF MICHIGAN, Ann Arbor, MI (US)

(72) Inventors: Nicolai Lehnert, Ann Arbor, MI (US); Hannah Palomino, Ann Arbor, MI (US)

(21) Appl. No.: 19/217,039

(22) Filed: May 23, 2025

Related U.S. Application Data
(60) Provisional application No. 63/651,623, filed on May 24, 2024.

(51) Int. Cl. B01J 31/06; B01J 37/00; B01J 37/32; B01J 37/36; C07K 14/795

(52) U.S. Cl. CPC B01J 31/062; B01J 37/0036; B01J 37/009; B01J 37/32; B01J 37/36; C07K 14/795

(57) ABSTRACT

A method of making heterogeneous biocatalysts can include admixing a protein dissolved in an aqueous buffer with an imprinting compound dissolved in a water-immiscible imprinting solvent, wherein the imprinting compound is hydrophobic and enters and remains in the active site of the protein due to hydrophobic interaction. A water-miscible organic solvent can then be added to precipitate the protein having the imprinting compound in the active site as an amorphous solid. The precipitate can be separated and lyophilized and then washed to remove the imprinting compound, thereby providing a heterogeneous biocatalyst.

### CROSS REFERENCE TO RELATED APPLICATION

The benefit of priority to U.S. Provisional Application No. 63/651,623 filed May 24, 2024, is hereby claimed and the disclosure is incorporated herein by reference in its entirety.

### BACKGROUND

### Field of the Disclosure

The disclosure relates to methods of making heterogeneous biocatalysts, and more particularly to methods of making heterogeneous biocatalyst with improved catalytic yield in organic solvents.

### Brief Description of Related Technology

Biocatalysts include both naturally occurring and engineered proteins and peptides. They have emerged as a class of tunable, selective, and biodegradable catalyst. Biocatalysts have capabilities as stereo-and enantioselective catalysts for complex chemical reactions. Biocatalysis has been successfully used in the late-stage functionalization of pharmaceuticals. Natural enzymatic catalysis typically takes place in aqueous solvents. Organic solvents are typically used, however, in industrial reactions. In such conditions enzymes are often insoluble or denature if mixtures of water and organic solvent are used, especially with increasing concentrations of organic cosolvent. These solubility and stability issues present significant hurdles for the use of some biocatalysts such as enzymes in industrial processes, limiting their application beyond aqueous reaction conditions.

There are three main methods for generating biocatalysts that operate in organic solvents: mutagenesis, immobilization, and heterogeneous biocatalysis. Mutagenesis and immobilization focus on facilitating catalysis in elevated concentrations of water-miscible organic co-solvent mixtures in monophasic systems. Heterogeneous biocatalysis relies upon the interplay between the protein's surface with the surrounding media. This interaction influences protein structure, dynamics, and catalysis. Proteins are dynamic and stable in aqueous environments which is facilitated by a hydration layer around the proteins' surface, and hydrogen bonding between amino acids on the surface and proximal water molecules. Consequently, adding moderate amounts of organic solvents can lead to denaturation due to disturbance of this hydrogen-bonding network. Heterogeneous biocatalysis can extend the techniques to water-immiscible organic solvents. However, the conventional approach suffers from low catalytic yields in chemical transformations compared to its aqueous counterpart.

There are limited examples of biocatalysts used in anhydrous organic solvents. Examples include lipase, Carlsberg subtilisin, horseradish peroxidase, and myoglobin. Lipase has been extensively studied in organic solvent enzymology, as this class of enzymes has the innate ability to exist in biological membranes which resemble aqueous/organic solvent mixtures.

Conventionally, biocatalysts have been prepared through direct lyophilization and precipitation. Direct lyophilization has been the most common way to remove aqueous solvent and rigidify the protein's structure. Precipitation is less common but has been used to strip the essential hydration layer when precipitating with polar organic solvents, thereby preserving the enzyme's structural integrity. To enhance enzymatic activity, preparatory methods have incorporated salt additives to help stabilize the enzyme structure, as well as inhibitor compounds to maintain the integrity of the active site. Despite these preparatory methods providing enzymes with unique biocatalytic attributes, such as thermostability, reusability, and switchable stereoselectivity, it remains that such methods result in low activity and catalytic yields when compared to similar reactions conducted under homogeneous conditions in aqueous media. This makes biocatalysts impractical for industrial use in organic solvent reactions.

### SUMMARY

Under certain organic solvent conditions, proteins exhibit rigidity and stability due to the diminished hydrogen bonding between protein and the bulk solvent, retaining only a few water molecules tightly bound to its surface, known as the essential hydration layer. Under conditions of limited hydration, the restricted protein dynamics inhibit denaturation. It has been observed that for efficient biocatalysis in both hydrophobic and hydrophilic organic solvents, a balance between the enzyme's essential hydration layer and the surrounding solvent is necessary. With this understanding, it has been found that proteins can be prepared as biocatalysts in an array of organic solvent reaction media.

Heterogeneous biocatalysts made by methods in accordance with the disclosure can include metalloenzymes that are natively found in aqueous media. Methods of the disclosure enable catalysis with these enzymes in anhydrous organic solvents. Significantly, methods of the disclosure enable efficient biocatalysis in organic solvents, with activity and yields comparable to the reactions in aqueous media. This unlocks the potential of biocatalysts as resources for industrial and academic synthetic applications that can significantly expand the scope of selective reactions and valuable compounds that can be obtained with biocatalysts.

### BRIEF DESCRIPTION OF THE DRAWINGS

FIG. 1 is a schematic illustration of a method in accordance with the disclosure.

FIG. 2 is a schematic illustration of a general cyclopropanation reaction used to screen activity of biocatalyst prepared by the method of the disclosure.

FIGS. 3A and 3B are graphs showing the results of the cyclopropanation using the biocatalysts prepared by the method of the disclosure. The reaction solvent in all cases was acetonitrile.

FIG. 4 is a graph showing results of cyclopropanation using the biocatalysts prepared in accordance with the disclosure as compared to biocatalysts prepared with no imprinting compound and to performance in aqueous solvent.

FIG. 5 is a graph showing cyclopropanation performance in different reaction solvents.

### DETAILED DESCRIPTION

Methods of forming heterogeneous biocatalysts in accordance with the disclosure include dissolving a protein having an active site in an aqueous buffer solution. Referring to FIG. 1, an imprinting compound is dissolved in a water-immiscible imprinting solvent and added to the aqueous buffer solution in which the protein is dissolved. Upon mixing, the imprinting compound enters the active site and remains in the active site. Without intending to be bound by theory, it is believed that the imprinting compound is drawn into and remains in the active site by hydrophobic interactions. The presence of the rigid imprinting compound in the active site stabilizes the active site, keeping it intact during precipitation. A water-miscible organic solvent is then added to the buffer solution to precipitate the protein having the imprinting compound in the active site as an amorphous solid. The precipitate is separated and lyophilized. After lyophilization, the precipitate is washed with a water-immiscible organic solvent to remove the imprinting compound and thereby form the solid heterogeneous biocatalyst. The chosen water-immiscible organic solvent is one in which the protein is not soluble.

It has advantageously been found that the methods of the disclosure, which rely upon a hydrophobic interaction of the imprinting compound with the active site, allow for retention of the imprinting compound in the active site to maintain the active site configuration during precipitation, but is easily removed after precipitation without adversely affecting the protein or its activity. This is in contrast to methods using inhibitor compounds. The methods of the disclosure can be free of inhibitor compounds.

The protein can be any protein of interest for biocatalysis reactions. The protein can be, for example, an enzyme. The enzyme can have a cofactor or can be an enzyme without a cofactor. For example, the protein can be metalloenzymes that are natively found in aqueous media. For example, the protein can be a heme protein. For example, the protein can be one or more of myoglobin, hemoglobin, neuroglobin, peroxidases, oxygenases, Cyt P450s, nitrophorin, nitrobindin, catalases, YfeX including I230A YfeX, and engineered heme proteins.

The imprinting compound is a hydrophobic, rigid compound. For example, the hydrophobic imprinting compound can be an aromatic and/or rigid cyclic compound. Other rigid hydrophobic compounds can also be used. For example, the imprinting compound can be a styrene, 2-vinylnapthalene, 9-vinylantrhacene, or betulin.

The aqueous buffer can be a buffer in which the protein is soluble. The protein can be dissolved in the aqueous buffer at room temperature. The protein concentration in the buffer can be about 10 mg/mL to about 50 mg/mL. For example, the protein concentration can be about 30 mg/mL. Examples of buffer include, but are not limited to, phosphate buffer, such as potassium phosphate buffer, tris (Hydroxymethyl) aminomethane. Use of other organic buffers is also contemplated herein.

The buffer can include a salt additive. The type and concentration of the salt additive can be selected according to the interaction with the essential hydration layer of the protein. The buffer and salts therein are selected to have a stabilizing effect on the protein. Salt additives can include NaCl and (NH₄)SO₄ and other inorganic salts. The salt additive can be present in the buffer at a concentration of about 0 M to 4 M, or about 0.01 M to 4 M. For example, the salt additive can be present in a concentration of about 300 mM. The buffer can alternatively be free of a salt additive.

The water-immiscible imprinting solvent can be any solvent suitable for dissolving the imprinting compound. For example, the solvent can be ethyl acetate, other esters and ethers, aromatic compounds, and the like. Selection of a suitable solvent for dissolving the imprinting compound can be made based on known properties of imprinting compounds and solvents. The imprinting compound can be present in a concentration of up to 1 M in the solvent or up to the solubility limit of the imprinting compound in the solvent.

The water-miscible solvent for precipitation can be a polar organic solvent. For example, the water-miscible solvent can be one or more of 1-propanol, 2-propanol, ethanol, acetone, and acetonitrile. The water-miscible solvent can be added to the admixture of the protein and the imprinting compound in a ratio of about 0.5:1 to about 6:1 organic solvent to buffer. For example, the ratio of the water-miscible solvent to aqueous buffer can be about 4:1.

Precipitation can be performed by adding the water-miscible solvent with constant stirring. For example, the precipitation can be performed while stirring at a rate of about 90 rpm. The water-miscible solvent can be added by rapid addition or dropwise addition.

The precipitate can be separated using any known separation methods. For example, the precipitate can be separated by centrifugation.

The separated precipitate can be lyophilized using known methods and conditions. The resulting dried pellet contains a solid heterogeneous biocatalyst. The biocatalysts can be washed to remove any excess imprinting compound. The pellet can optionally be pulverized prior to washing.

The lyophilized protein can be washed with a water-miscible washing organic solvent. Examples include, but are not limited to, 1-propanol, 2-propanol, ethanol, acetone, and acetonitrile, and other precipitation solvents mentioned above. The washing organic solvent can be any organic solvent that does not dissolve the protein.

Methods of the disclosure can include incorporating the imprinting compound through admixture of the dissolved imprinting compound and the dissolved protein at room temperature. Precipitation can also be performed at room temperature.

Methods of the disclosure were used with myoglobin (Mb) as a pilot model biocatalyst. Mb's biological function is dioxygen storage; however, it has an abiological carbene transfer reactivity that has been extensively studied in traditional aqueous biocatalysis. As a mode of comparison, the cyclopropanation reaction was used, due to its often low substrate solubility in water and its prevalence as a stereoisomeric pharmaceutical motif.

### EXAMPLE

### Biocatalyst Preparation

Mb was initially dialyzed in milliQ water for 24 hours, followed by lyophilization for 24 hours. The initial buffer-protein solution was obtained by adding 5 mg of lyophilized Mb to 167 μL of pH 7.4 50 mM Kpi buffer solution, containing either 300 mM NaCl, 300 mM (NH₄)₂SO₄ or no additional salt. The solution was gently mixed at room temperature (RT) for 5 minutes in a 15 mL glass vial. After Mb was dissolved, 20 mM of styrene was added from a 1 M stock made in ethyl acetate. The buffer-protein-imprint solutions were stirred for another 5 min at RT and then precipitated with one of the following 5 polar organic solvents, 1-propanol, 2-propanol, ethanol, acetone, or acetonitrile, in a 4:1 ratio of organic solvent to buffer. Each precipitation combination was tested with a dropwise and rapid organic solvent addition rate (2 min and less than 5 seconds addition, respectively). A stir rate of 90 rpm was maintained during the organic solvent addition and for 30 minutes following to ensure full aqueous-organic solvent mixing and precipitation. Interestingly, each condition gave varying appearances of the protein precipitates along with varying rates of precipitation, which was attributed to the differing interactions with the essential hydration layer that dictate aggregation.

The resulting precipitate was centrifuged at 2000 rpm to separate the supernatant. The protein pellet was lyophilized for 24 hours, then subjected to manual crushing with a mortar and pestle, followed by ×500 μL washes of the powder with the respective reaction solvent to remove any excess imprinting compound.

The method of the disclosure was performed varying the conditions of the buffer (salt additive) and the precipitation conditions. Two different salts were selected, NaCl and (NH₄)₂SO₄, according to their interactions with the essential hydration layer of Mb and the stabilizing effects they provide. Without intending to be bound by theory, it was believed that using these salts would enhance precipitation by stabilizing the structures. Altogether three different buffer conditions were tested; pH 7.4 potassium phosphate buffer with 300 mM of NaCl, 300 mM of (NH₄)₂SO₄ and no salt additive. Initially, 5 mg of Mb was combined with buffer at room temperature (RT) in a 15 mL glass vial at a concentration of 30 mg/mL.

It was observed that the heterogeneous preparations of Mb can be resuspended in aqueous buffer with structural recovery and conservation of relative heme content, as determined by UV-vis and CD spectroscopy, demonstrating that the method of the disclosure using the imprinting compound modified the structural conformation in a non-deleterious way.

### Optimization with Different Imprinting Compounds

In an effort to increase the yields, the cyclopropane product, (1R,2R)-2-phenyl-cyclopropanecarboxylic acid ethyl ester, was used as the imprinting compound in place of styrene. Ethyl acetate as the imprinting solvent was used, as the cyclopropane product fully precipitated from solution when added with buffer or with methanol. The cyclopropane product was initially used as the imprint, but curiously, a 0% yield was observed in the cyclopropanation reactions detailed below. The absence of the EDA dimer peak was also noted, suggesting that the imprint was not entering the active site of Mb. Several other conditions with 1-propanol precipitation were tested and it was found that the rapid addition, NaCl additive, 1-propanol precipitation gave a 40% cyclopropanation yield when using the cyclopropane imprint. Without intending to be bound by theory, it is believed that the precipitation solvent must be selected with consideration of an affinity gradient between the active site and the precipitation solvent for driving these non-inhibitor imprinting compounds into the active site of Mb.

### Cyclopropanation Reactions

Cyclopropanation reactions for each set of conditions were performed using 1 mg (90 μM) of precipitated Mb in 500 μL of anhydrous acetonitrile with 2 mM of cobaltocene as the reductant, along with 20 mM of ethyl diazoacetate and 20 mM of styrene as the substrates. Triplicate reactions were run for 16 hours at RT with high-speed stirring (˜200 rpm) under anaerobic conditions. The product was easily isolated by centrifuging the suspension with a tabletop centrifuge for 2 minutes to separate the solid biocatalyst. To ensure that the styrene imprint was fully washed out, control reactions excluding styrene were run, which gave no cyclopropanation yields, demonstrating that after workup, no additional styrene imprint remains. The yields for each reaction were determined using Gas Chromatography Mass Spectroscopy (GC-MS). The enantiomers were quantified using Supercritical Fluid Chromatography (SFC).

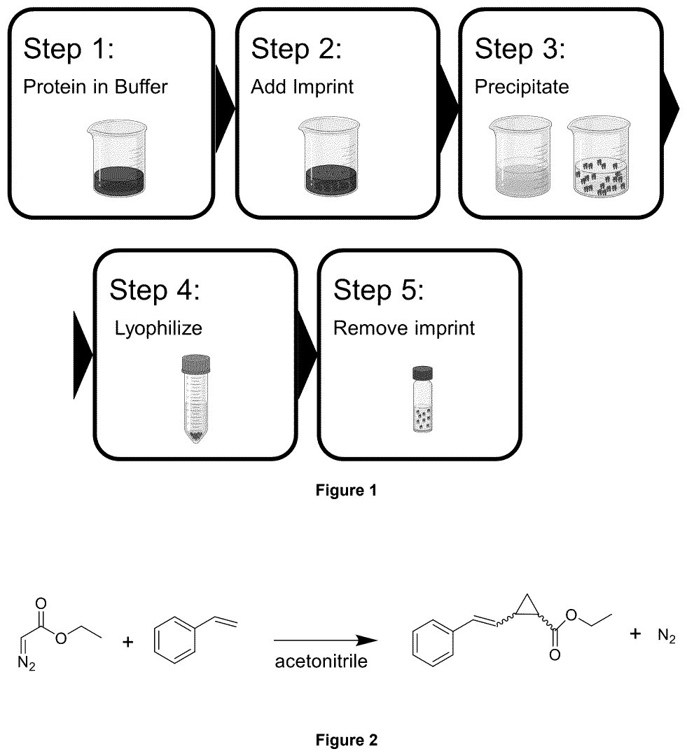

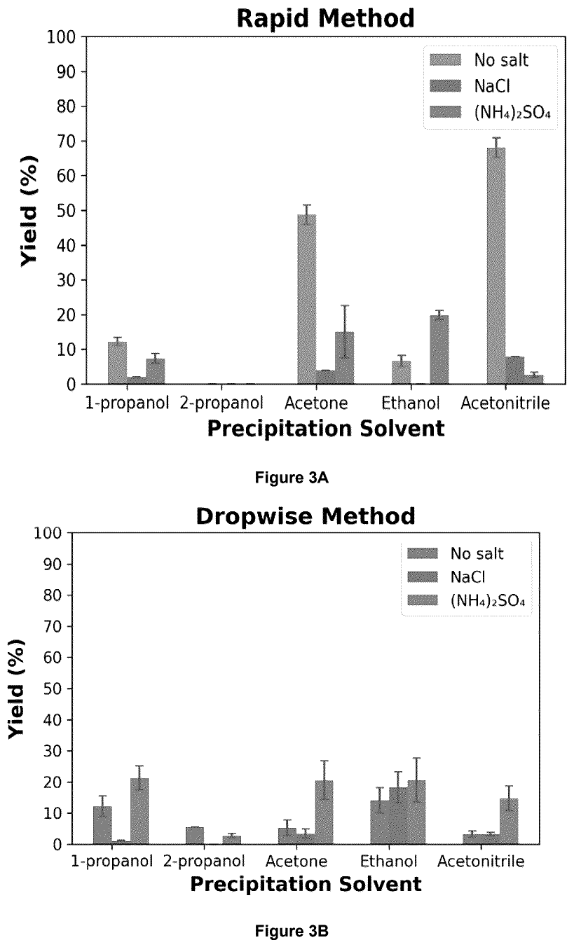

Referring to FIG. 2, cyclopropanation reactions were initially performed in acetonitrile to test the effectiveness of each precipitation condition for heterogeneous preparations of Mb. Referring to FIGS. 3A and 3B, it was observed that buffer conditions containing NaCl, regardless of the precipitating solvent or the rate of addition, resulted in cyclopropanation yields lower than 10%, except for ethanol added dropwise, which gave 18% yield. These results suggest that the stabilizing effect observed with NaCl was non-preferential for maintaining the structural integrity during precipitation. Interestingly, buffer conditions containing (NH₄)₂SO₄ increased reaction yields to about 15-21% for all precipitating solvents except 2-propanol with dropwise addition. This trend matched the hypothesis that the rigidifying effects of (NH₄)₂SO₄ would help with the precipitation step. Interestingly, the cyclopropanation yields were slightly lower overall with rapid precipitation. Precipitation conditions with no salt resulted in the greatest deviation in yields across the precipitating solvents and addition rates. Using a dropwise precipitation with no salt additive, a 45% cyclopropanation yield was observed using 1-propanol while the other solvents under these conditions produced less than a 15% yield. Highest yields were obtained under the no salt, rapid precipitation conditions, while 1-propanol, 2-propanol, and ethanol preparations all produced yields lower than 20%. A 45% yield was observed using acetone and, excitingly, the highest cyclopropanation yield of 68% was obtained with acetonitrile.

These results highlight the delicate interplay between the ions, solvent, and the protein, displaying more consistency between the different precipitation solvents when higher concentrations of ions are present. The reusability of the heterogeneous protein catalyst was tested three times. Notably, no significant difference was observed after the second use (64% cyclopropanation yield) but observed a 41% decrease after the third use (33% yield).

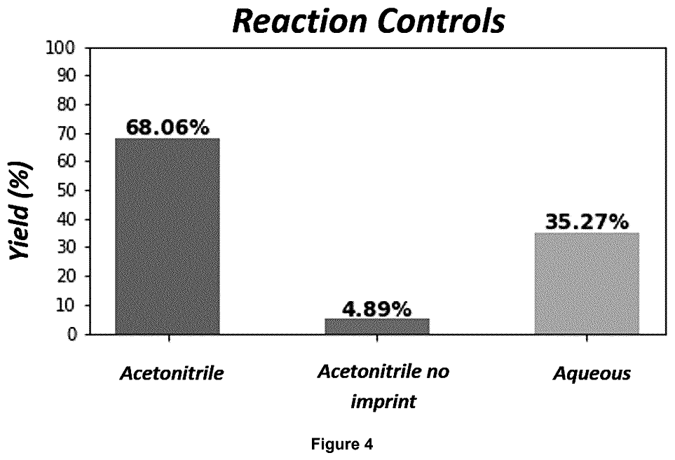

Referring to FIG. 4, precipitation without the styrene imprint was performed to illustrate the significance of the imprinting compound in improving yield. The biocatalysts precipitated without the styrene imprint resulted in an average cyclopropanation yield of only 4%. Furthermore, when the heme was accessible in the active site of Mb, the dimerization of EDA was observed, especially when the olefin component was not present in the reaction. EDA dimerization therefore serves as an additional indicator of active site accessibility in these test reactions. However, in the absence of the imprinting compound (styrene) during protein precipitation, negligible EDA dimerization was observed, indicating that EDA is not able to interact with the heme. Therefore, the styrene imprint was demonstrated to be effective in preserving the active site in an accessible conformation with the methods of the disclosure.

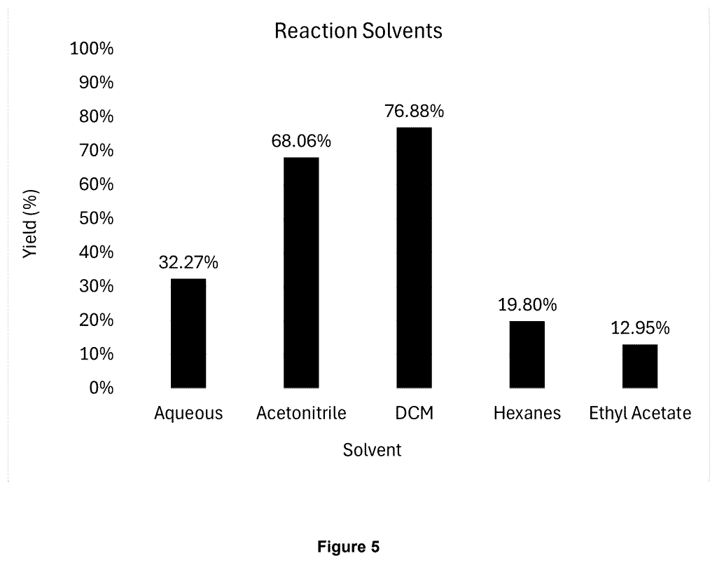

While the protein concentration employed for heterogeneous reactivity exceeds that typically utilized in traditional biocatalysis by approximately 2-3 times, a comparison of the reactivity with Mb in an aqueous buffer as a homogeneous catalyst was conducted. Referring to FIG. 5, despite the diffusional limitations inherent with heterogeneous catalysis due to the presence of particles, it was observed that the methods of the disclosure resulted in a heterogeneous biocatalyst that gave a 68% yield for the cyclopropanation of styrene with EDA in acetonitrile. This yield significantly surpasses the 32% cyclopropanation yield obtained with Mb at an equivalent concentration in a buffer solution. Such results were surprising and unexpected, as there is no reported instance where a biocatalyst has demonstrated superior yields in heterogeneous phase in organic solvents compared to the same reaction in homogeneous phase (i.e., aqueous media).

With the methods of the disclosure, Mb was demonstrated to efficiently perform cyclopropanation reactions in a variety of organic solvents. Most notably, a 78% cyclopropanation yield was observed in dichloromethane as the reaction solvent, one of the most used organic solvents in organic synthesis. A 36% yield was achieved in hexanes, a solvent which had not previously shown reactivity with Mb. A 13% cyclopropanation yield was achieved in ethyl acetate. No yields were observed in benzene and toluene. Without intending to be bound by theory, it is believed that the precipitation conditions can be tuned and/or tailored to lead to different catalytic abilities in other solvents.

### Methods of the Disclosure using Pharmaceutically Relevant Substrates

Using the logP scale where compounds with a logP value of greater than 2 are considered practically water insoluble, substrates along that range were selected. Styrene as a standard substrate has a logP of 2.96. 2-vinylnaphthalene, 9-vinylanthracene and betulin which have logP values of 4.23, 5.50 and 9.71, respectively, were also tested. The method of the disclosure as described above was performed using these various imprinting compounds. It was found that 2-vinylnaphthalene imprinted well under the tested conditions and gave a 46% cyclopropanation yield in the reaction of 2-vinylnaphthalene with EDA in acetonitrile. When Mb was imprinted with 9-vinylanthracene, insignificant cyclopropanation yields were observed and a much weaker dimerized EDA peak was observed, signifying that this compound did not imprint well under the standard conditions. However, when 2-vinylnaphthalene-imprinted Mb was used to perform the 9-vinylanthracene reactions, a 23% cyclopropanation yield was observed. Precipitation conditions with 1-propanol were investigated as well and no yield was observed.

The use of heterogeneous Mb to perform the cyclopropanation reaction on betulin, a steroidal compound used as a pharmaceutical backbone, was evaluated to determine whether the alkene attached to ring 5 of the compound could be functionalized. Betulin has a logP of 9.71 and is poorly soluble in ethyl acetate; therefore, imprinting was performed with a lower concentration of this compound. Acetonitrile precipitation conditions were used; however, due to solubility issues in acetonitrile, the cyclopropanation reaction was performed in ethyl acetate. While the product could not be fully detected and quantified with GC-MS, 2D NMR was used to determine the presence of the cyclopropane motif in the reaction mixture, resulting from addition of EDA to the vinyl group of ring 5 of betulin.

### YfeX Biocatalyst Preparation

The initial buffer-protein solution was obtained by adding 180 μL of 80 μM I230A YfeX to 50 mM Kpi buffer solution at pH 7.4, containing 300 mM NaCl. The solution was gently mixed at room temperature (RT) for 5 minutes in a 15 mL glass vial. Once I230A YfeX was dissolved, 20 mM of styrene were added from a 1M stock made in ethyl acetate. The buffer-protein-imprint solution was stirred for another 5 min at RT and the precipitated with acetonitrile at an 8:1 ratio of organic solvent to buffer using a “rapid” rate of addition. A stir rate of 90 rpm was maintained during the organic solvent addition and for the 30 min following to ensure full aqueous-organic solvent mixing and precipitation.

The resulting precipitate was centrifuged at 2000 rpm to separate the supernatant. The protein pellet was lyophilized for 24 hours, then subjected to manual crushing with a spatula, followed by ×500 μL washes of the powder with the reaction solvent (i.e., acetonitrile) to remove any excess imprinting compound.

### YfeX Cyclopropanation Reactions

Cyclopropanation reactions for each set of conditions were performed using ˜1 mg/360 μL precipitated and lyophilized I230A YfeX (slight variations in weight are attributed to varying salt concentrations in the buffer), i.e., ˜0.0288 μmol in 500 μL of anhydrous acetonitrile with 2 mM of cobaltocene as the reductant, along with 20 mM of ethyl diazoacetate and 20 mM of styrene as the substrates.

Triplicate reactions were run for 16 hr at RT with high-speed stirring (˜200 rpm) under anaerobic conditions. The product was easily isolated by centrifuging the suspension with a tabletop centrifuge for 2 min to separate the solid biocatalyst. To ensure that the styrene imprint was fully washed out, control reactions excluding styrene were run, which gave no cyclopropanation yields, demonstrating that after workup, no additional styrene imprint remains. An average yield of 52±3.5% was obtained. The yields for each reaction were determined using Gas Chromatography Mass Spectroscopy (GC-MS).

The foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In the case of conflict, the present description, including definitions, will control.

Throughout the specification, where the compounds, compositions, methods, and/or processes are described as including components, steps, or materials, it is contemplated that the compounds, compositions, methods, and/or processes can also comprise, consist essentially of, or consist of any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

What is claimed is:

1. A method of forming heterogeneous biocatalysts, comprising:
dissolving a protein having an active site in an aqueous buffer solution;
adding an imprinting compound dissolved in a water-immiscible imprinting solvent to the buffer solution, wherein the imprinting compound is a hydrophobic compound and enters the active site and remains in the active site due to hydrophobic interaction;
adding a water-miscible organic solvent to the buffer solution to precipitate the protein having the imprinting compound in the active site as an amorphous solid;
separating the precipitate;
lyophilizing the precipitate; and
washing the precipitate with a washing water-immiscible organic solvent to remove the imprinting compound and thereby forming the heterogeneous biocatalyst, wherein the protein is not soluble in the washing organic solvent.

2. The method of claim 1, further comprising crushing the lyophilized precipitate into a powder before washing.

3. The method of claim 1, wherein the buffer solution comprises potassium phosphate buffer and/or tris (Hydroxymethyl) aminomethane.

4. The method of claim 1, wherein the buffer solution is free of salt.

5. The method of claim 1, wherein the buffer solution comprises a salt.

6. The method of claim 5, wherein the salt is present in a concentration of up to about 4M or 0.01M to 4M.

7. The method of claim 5, wherein the salt is NaCl or (NH₄)₂SO₄.

8. The method of claim 1, wherein the imprinting compound is a styrene, 2-vinylnapthalene, 9-vinylantrhacene, or betulin.

9. The method of claim 1, wherein the imprinting compound is added in an amount of up to about 1M or 0.01M to 1M.

10. The method of claim 1, wherein the imprinting solvent can comprise esters, ethers, aromatic compounds, or the imprinting solvent is ethyl acetate.

11. The method of claim 1, wherein the water-miscible organic solvent is added to the buffer solution comprising the protein having the imprinting compound in the active site is one or more of alcohols, ketones, and nitriles, or the organic solvent is one or more of 1-propanol, 2-propanol, ethanol, acetone, and acetonitrile.

12. The method of claim 1, wherein the water-miscible organic solvent is added to the buffer solution in a ratio of about 0.5:1 to 6:1 organic solvent to buffer.

13. The method of claim 1, comprising adding the water-miscible organic solvent to the buffer solution as a single rapid addition to precipitate the protein.

14. The method of claim 1, wherein the protein is a protein with a cofactor, for example a heme protein.

15. The method of claim 14, wherein the heme protein is myoglobin, hemoglobin, neuroglobin, peroxidases, oxygenases, Cyt P450s, nitrophorin, nitrobindin, catalases, YfeX, and engineered heme proteins.

16. The method of claim 1, wherein the washing organic solvent is 1-propanol, 2-propanol, ethanol, acetone, acetonitrile.
